(19) United States
(12) Patent Application Publication
(10) Pub. No.: US 2007/0276454 A1
(43) Pub. Date: Nov. 29, 2007

(54) Apparatus for Illuminating a Zone of Mammalian Skin

(76) Inventor: Christopher Edwin Williams, Llanelli (GB)

Correspondence Address: Fitzgerald, John K.;Fulwider Patton, Howard Hughes Center, 6060 Center Drive, Tenth Floor, Los Angeles, CA, 90045, US

(21) Appl. No.: 10/575,588

(22) PCT Filed: Oct. 15, 2004
(86) PCT No.: PCT/GB04/04362

§ 371 Date: Mar. 28, 2007

(30) Foreign Application Priority Data
Oct. 16, 2003 (GB) 0324254.2

(51) Int. Cl. A61N 5/06

(52) U.S. Cl. 607/88

(57) ABSTRACT

Apparatus for illuminating a zone of mammalian skin which comprises one or more light emitters (3). An applicator such as a suction cup (5) or an adhesive patch can be moved to direct light to the skin and a controller can be used to control the duration and/or intensity of light delivered without significant ablation of the skin. The apparatus is used in method for cosmetically improving the appearance of skin.

The present invention relates to an apparatus and method for illuminating a zone of mammalian skin.

It is well known to utilize many forms of light to improve the appearance of skin. Among lighter-skinned people, for example, ultra-violet light has been used to produce a tan, and to otherwise improve general skin color and tone. In many cultures light has been used to reduce acne and other skin blemishes. Light has also been used medically to stimulate wound healing, and to treat inflammatory conditions, skin ulcers, and wrinkles.

Acne Vulgaris is a condition of the sebaceous glands which affects 80% of individuals between the ages of 11 and 30. It is not confined to these age groups however and can affect all ages from neonates to the elderly. Factors which are considered of primary significance to the condition include an increase in the production of sebum, abnormal follicular keratinisation, the presence of Proprionibacterium and subsequent inflammation.

Dependant on the size, content, and depth of the inflamed acne lesion, it is defined as a papule (less than 0.5 cms in diameter), nodule (elevated solid lesion > than 0.5 cms) pustule (a papule what contains purulent material) or a cyst (nodule that contains fluid or semisolid matter).

Hair follicles are minute passages in the skin, which allow hairs to grow and produce sebum secretions from sebaceous glands which are housed within the hair follicle. Due to increased androgen levels or an excessive reaction by the sebaceous glands to androgen production, the sebaceous glands enlarge resulting in increased secretion of sebum which along with the keratinisation process of the epithelial cells, clog the hair follicle. Initially these blockages are microscopic then develop into whiteheads or blackheads (Comedones). Congested follicles are an ideal medium for growth of bacteria. When sebum levels are increased, the skin commensal proprionibacterium ingest the clogged sebum under the skin and chemicals are produced which trigger the immune system to initiate the inflammatory changes and erythematous macules associated with Acne Vulgaris.

Inflammation is the body's response to invasion of pathogens and the redness associated with the acne lesions is the result of increased blood flow whereby the white blood cells invade bacterial cells and damage tissue and produce pus. Other fluids flood to the area and collect at the site of the inflamed tissue.

The approaches which are currently used for tackling Acne Vulgaris include Drug therapies (Systemic antibiotics, cortisone injections, Dianette (women only contraceptive pill, Roaccutane, Retinoids), PUVA (Psoralen and Ultra Violet light, type A), UVB Phototherapy, Dermalux (a system using a combination of red and blue light to treat acne), Peeling agents, laser resurfacing, Dermabrasion and Microdermabrasion.

Known apparatus for treating acne by irradiation are disclosed, for example, in GB2356570, WO00/32272, WO02/32505 and U.S. Pat. No. 5,549,660. These apparatus are typically large and cumbersome and require a subject to visit a surgery for treatment.

There is a continuing need for an apparatus and method which allows administration of a low safe dose of illuminating radiation for individual need away from a doctors or cosmetic surgery.

The present invention provides an improved apparatus and method for illuminating a zone of mammalian skin.

According to the present invention, there is provided an apparatus for illuminating a zone of mammalian skin, the apparatus comprising:
a) a light emitter;
b) an applicator movable to direct light emitted from the light emitter to the zone of mammalian skin; and
c) a controller controlling the duration and/or intensity of light delivered to the surface of the skin to provide a desired effect without producing significant ablation of the mammalian skin.

In the context of the invention, significant ablation of the skin may comprise a substantially deleterious effect upon a basal layer of skin. More particularly, “without producing significant ablation” preferably means that no more than 5% of the cells in the area of skin being irradiated die within 5 hours of the application of the light. It is even more preferable that no more than 3%, or even 1% of such cells die within the 5 hour window after such application and even more preferred that less than 1% of such cells die.

As used herein the term “light” is not limited to visible light. Also contemplated are other wavelengths of light including especially near UV and near IR. The term “light” is thus synonymous with the term “electromagnetic radiation”.

The applicator is preferably adapted for removable attachment to the zone of mammalian skin. In a preferred embodiment the applicator comprises a suction cup and adhesion to the skin may be facilitated by provision of an adhesive, such as a replaceable or consumable hypo-allergenic adhesive ring. Alternatively the applicator may be an adhesive patch for example a double sided adhesive patch to which the light emitter is attached and then the patch is applied to a body surface such as the skin or teeth. The inner surface of the suction cup is preferably of a light reflecting material. The light reflective material may be a silver or white coating or lining on the inside surface of the cup.

The adhesive may include an active material such as a topical agent that can be activated by light emitted to act on the body surface.

The apparatus preferably includes a plurality of light emitters. Preferred light emitters are light emitting diodes (LEDs) and diode lasers. The apparatus may use other light fluence sources. In the case of LEDs a single LED or a number of LEDs for example a bank of LEDs may be used. The wavelength of light emitted from the light emitter is preferably substantially in the range 400 nm to 1000 nm, more preferably substantially in the range 400 nm to 450 nm and/or 570 nm to 590 nm, even more preferably substantially in the range 570 nm to 590 nm. When a plurality of light emitters are provided in accordance with a preferred embodiment of the present invention, one or more of the light emitters may emit light in a predetermined wavelength range and one or more different light emitters may emit light in a different predetermined wavelength range. For example, one or more light emitters may emit blue light (400 nm to 500 nm) and one or more light emitters may emit yellow light (570 nm to 590 nm). In a particularly preferred embodiment of the present invention, the light emitter is received in the applicator.

The controller is preferably provided in a housing which may also include a power source. The power source is preferably one or more batteries, typically one or more rechargeable batteries. The housing is ideally a small, light, portable, pocket sized unit, typically similar in size to a personal stereo, which can be easily carried by a user, for example strapped to a belt or inserted in a pocket The user is therefore, advantageously free to move about whilst receiving treatment from the apparatus.

The energy density of light directed toward the zone of mammalian skin is typically low intensity to avoid significant ablation of the mammalian skin. This provides that the apparatus is suitable for personal use away from a doctors or cosmetic surgery.

The controller is preferably configured to control the period of time that light is emitted from the light emitter for one treatment period. The duration of light emitted from the light emitter is preferably substantially in the range 1 minute to 1 hour, more preferably substantially in the range 20 to 40 minutes, more preferably, about 30 minutes. Desirably, the controller is configured to permit variable selection of light duration within the preferred range and/or inhibit the duration of the period of light emitted above substantially 1 hour.

The light emitted may be pulsed or continuous wave. Pulsed energy may be preferred in order to avoid overheating of the skin whilst producing the desired effect. Also by using pulsed operation, light emitting devices (particularly LED's) may be driven harder to produce more light output. A typical LED can operate at a drive current of 50 mA in continuous mode, whilst in pulsed operation, for short periods, the same diode can be pulsed at current of around 200 mA. Pulsed operation will also allow more diodes to be used for a given energy source requirement, thus reducing the power requirement and prolonging the life of the battery or the like used to power the light emitters.

According to a further aspect, the present invention provides a method of cosmetically improving the appearance of skin, the method comprising providing apparatus according to the present invention and directing light of a predetermined wavelength toward a target area of skin in accordance with a predetermined delivery regime.

The method of the present invention is preferably used for the treatment of acne vulgaris.

The method according to the present invention provides a low intensity light and is therefore suitable for home use away from a doctors or cosmetic surgery without any medical intervention.

As hereinbefore described in connection with the apparatus of the present invention, the term “light” is not limited to visible light. Also contemplated are other wavelengths of light including especially near UV and near IR. The term “light” is thus synonymous with the term “electromagnetic radiation”.

The period that the light is directed towards the skin is preferably substantially in the range 1 minute to 1 hour, more preferably 20 to 40 minutes, more preferably about 30 minutes.

The wavelength of light directed towards the skin surface is preferably substantially in the range 400 nm to 1000 nm, more preferably substantially in the range 400 nm to 450 nm and/or 570 nm to 590 nm, most preferably in the range 570 nm to 590 nm.

The light directed towards the skin surface may be pulsed or continuous wave. Pulsed energy may be preferred in order to avoid overheating of the skin whilst producing the desired effect.

When the apparatus or method of the present invention is used for the cosmetic treatment of a skin condition (particularly acne vulgaris), the light directed to the zone of mammalian skin typically is of a predetermined wavelength and is delivered according to a predetermined regime in order to have the desired effect at the zone of skin including a reaction leading to at least partial disabling or eradication of the cause of the skin condition.

A feature of the technique of the invention is that efficacy is achieved without the need of any other topically applied agent or any invasive or ablative procedure. However, it is also contemplated that the apparatus of the present invention could be used in conjunction with a topical agent (such as a retanoid) applied to the skin.

The radiation is typically low intensity (avoiding ablation at or below the skin surface) and typically primarily of wavelength at or about the wavelength of yellow light (570 nm to 590 nm) for reasons explained in detail later. Absorption of light is through the dermal vasculature having no adverse effects on the epidermis.

Desirably, the reaction leading to at least partial removal or disabling of the cause of the skin condition is a photochemical reaction, caused by selective absorption of the predetermined wavelength light, typically by a preselected chromophore.

For Acne Vulgaris the chromophore targeted to combat the proprionibacterium is porphyrin in the connective tissue. This tissue bound photosensitiser when excited from light of a certain wavelength (typically the wavelength of blue light 400 nm to 450 nm and yellow light 570 nm to 590 nm), produces a photochemical reaction resulting in the production of singlet oxygen thereby destroying the bacterium.

Proprionibacterium is averse to oxygen (anaerobic) and relies upon chemicals known as porphyrins in skin tissue. Porphyrin is usually innocuous in the absence of light. It is however photosensitive and when exposed to light of the required wavelength the photochemical reaction occurs. This results in a transition from the porphyrin's ground state to a reactive triplet state. At this level, a reaction with molecular oxygen creates singlet oxygen. Through the medium of a suitable light source, to activate the porphyrins to produce singlet oxygen, the bacterium responsible for Acne Vulgaris can be cleared in a cosmetic, pain-free, non-invasive and efficient manner.

Vasodilation and hyperaemia are integral parts of the inflammatory response, including response to infection. Therefore any inflammatory/infective focus contains a disproportionate concentration of red blood cells.

Porphyrin molecules are contained in the heam of haemoglobin so that any inflammatory or infective focus contains a concentration of natural porphyrin. Activation of this porphyrin using, for example, yellow (570 nm to 590 nm) light releases substances which destroys adjacent toxins such as bacteria in acne.

Similarly, any acute inflammatory condition of skin such as rosaceae will be helped although the exact toxin may be unknown.

Yellow light (570 nm to 590 nm) is beneficially readily absorbed by the epidermis and will generally penetrate the tissue underlying the epidermis to reach the target porphyrin.

Where the skin condition is Acne Vulgaris it is therefore preferred that the wavelength of the illuminating radiation comprises a primary wavelength or narrow wavelength band substantially in the range 570-590 nm.

The photochemical interaction is typically dependent upon the number of incident photons, so the photons may be delivered in pulsed or continuous wave mode.

The target for the light has to be a material that absorbs a specific wavelength and disregards other wavelengths (chromophore). In accordance with the invention, for Acne Vulgaris, the chromophores may be porphyrin in skin tissue and oxyhaemoglobin in the dermal vasculature.

The preferred wavelength (or wavelengths) for this invention will depend upon the skin condition being treated but typically include a wavelength in the range of 400 nm to 1500 nm with a preferred range of 400 nm to 450 nm and/or 570 nm to 590 nm, and even more preferred range of 570 to 590 nm.

The invention will now be further described in an exemplary embodiments, by way of example only, with reference to the accompanying drawings in which:

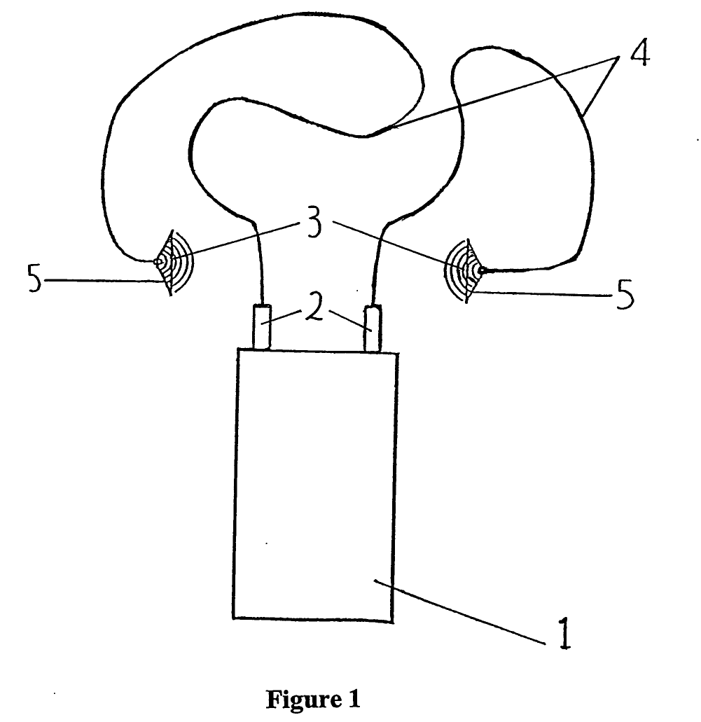

FIG. 1 is a schematic view of an exemplary embodiment of the apparatus of the present invention; and

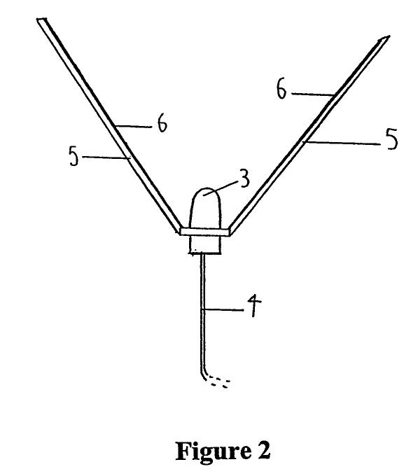

FIG. 2 is a schematic view of part of the apparatus of FIG. 1.

The apparatus shown in FIG. 1 comprises a base unit 1 with four remote light emitters 3 (only two light emitters are shown) connected to the base unit 1 via electrical or optical leads 4.

The base unit 1 can be of any suitable size, shape, colour, material etc. It is preferred however, that the base unit 1 is a small, light, portable pocket sized unit, for example similar in size to a personal stereo. A user would therefore advantageously be able to use the apparatus at home for extended periods at their convenience, whilst moving about the home.

The base unit 1 houses a controller which regulates the duration and/or intensity of light emitted from the light emitters 3 to produce a desired effect without producing significant ablation of the mammalian skin.

A fixed resistor (not shown) may be provided to limit the current drawn by light emitters 3 and thus reduce the power required.

The controller may be a timer provided in the base unit 1 which can be set to provide output of light from the light emitters 3 for a predetermined period of time. By providing a low energy density of light for a long duration (typically 30 minutes) a desired effect, for example cosmetic treatment of a skin condition such as acne, typically results without producing significant ablation of the mammalian skin. The light emitted may be in a continuous wave or pulsed to improve absorption by allowing thermal relaxation periods in the treatment. The controller typically regulates the pulse width and duration according to a predetermined regime.

A user may desirably be able to set the controller such that the light emitted is of an energy density, duration and pulse regime according to the desired effect required and the users skin type (for example, fair or dark skinned). Alternatively, or additionally, the controller may be able to automatically set the optimal parameters typically following some input from the user, such as desired effect required and skin type. Whatever parameters are selected whether automatically or manually, the controller is suitably configured to only allow light emitted to be of a wavelength, energy density, duration and pulse regime that will not produce significant ablation of the users skin, preferably substantially no ablation. This provides that the apparatus is suitable for personal use away from a doctors or cosmetic surgery.

The base unit 1 may also house a power source. The power source can be of any known type, conventional or otherwise and is preferably one or more batteries which are ideally rechargeable.

The light emitters 3 may be light emitting diodes (LEDs) or diode lasers. Where lasers are used, the output may be monochromatic. Alternatively, or in the case where LED's are used, the radiation delivered may be “effectively” monochromatic (for example by means of appropriate filtering) or of a relatively narrow band width (typically within a band width of 15 nm of less).

Preferably the light emitters 3 are LED's either in the blue, green, yellow or red spectrum, preferably the blue or yellow spectrum, most preferably the yellow spectrum. One or more of the light emitters 3 may emit light of first spectrum (for example, blue) whilst the other light emitters 3 may emit light of a second spectrum (for example, yellow) different from the first spectrum. The light emitters 3 are each connected to the base unit 1 by electrical or optical leads 4. The leads 4 have a plug 2 at one end and a suction cup 5 at the other end. The plug 2 can be removably plugged into base unit 1, so that a user can plug between one and four light emitters 3 into the base unit 1 depending on the treatment required. The light emitters 3 are each housed in the suction cup 5 attached to the end of the leads 4, as shown more clearly in FIG. 2.

The inner surface of the suction cup 5 is lined with a reflective material 6, such as a silver or white lining. The reflective material acts to increase the light directed to the surface of the mammalian skin.

In use, one or more of the suction cups 5 can be attached to the surface of a users skin at any location of the body where treatment is required. For example, the suction cup 5 can be attached in the proximity of a spot to bathe the area in a controlled intensity of light for a predetermined period of time. Attachment to the skin can be facilitated by an adhesive, such as a replaceable or consumable hypoallergenic adhesive ring.

However, it is envisaged that rather than using suction cups, the light source can be attached to the skin by way of an adhesive which is part of an adhesive patch. The light source may be attached to the adhesive or a carrier for the adhesive. Alternatively the light source may be housed within the body of an adhesive patch. For example the light source may be included in a layer of for example a plaster which can then be attached to the skin. This arrangement has particular advantages for attaching to profiled body parts such as the face as it provides a flexible member that can be secured so that the member follows the contours of the face. The light source therefore can act efficiently as it is held in close proximity to the skin.

The presently disclosed apparatus and methods have numerous applications that improve the appearance of skin, that is having a desired skin effect. Where ultra-violet light is used, for example, the improvement may involve tanning. Where light is applied at 585 nm, or other wavelength that is well absorbed by the dermis, but only poorly absorbed by the epidermis or basal layer, the improvement in skin may be in making the skin smoother. Smoother skin can be achieved, for example, through a reduction in the depth or width of a wrinkle, or through increasing collagen production under an indentation caused by acne. Another improvement may be treatment of acne, particularly acne vulgaris. Another improvement may be reduction in the growth or presence of hair, such as where the application conditions are satisfactory to kill or at least significantly damage cells structurally and/or physiologically associated with growth of hair, and particularly cortical cells including hair follicle cells, papilla cells, outer and inner root sheath cells (e.g.: Huxley's layer and Henley's layer). Still another improvement in skin can be achieved indirectly, by affecting a medical condition, such as the treatment of skin cancers. The apparatus may be used in conjunction with a photodynamic therapy (PDT) drug, typically applied to the surface of the skin where the light is directed.

The apparatus of the inventions has a wide range of applications for affecting skin conditions such as acne which may be as a result of infection with acne vulgaris. The apparatus may be used to treat more deep seated skin conditions such as ulcers or skin lesions. However, the apparatus may be used in methods of treating the skin or body which have purely cosmetic applications such as reducing wrinkles, evening out skin tone or even the whitening of teeth.

Specific embodiments and applications of light therapy methods and apparatus have been disclosed. It should be apparent, however, to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context.

1. An apparatus for illuminating a zone of mammalian skin, the apparatus comprising:
a) a light emitter;
b) an applicator movable to direct light emitted from the light emitter to the zone of mammalian skin; and
c) a controller controlling the duration and/or intensity of light delivered to the surface of the skin to provide a desired effect without producing significant ablation of the mammalian skin.

2. Apparatus according to claim 1, wherein, the applicator is adapted for removable attachment to the zone of mammalian skin.

3. Apparatus according to claim 2, wherein the applicator comprises a suction cup.

4. Apparatus according to claim 3, wherein adhesion of the suction cup to the skin is facilitated by provision of an adhesive, such as a replaceable or consumable hypo-allergenic adhesive ring.

5. Apparatus according to claim 3 or 4, wherein the inner surface of the suction cup is of a light reflecting material.

6. Apparatus according to claim 1 or claim 2 wherein the applicator comprises an adhesive layer.

7. Apparatus according to claim 6, wherein the light emitter is attached to or enclosed within the adhesive layer.

8. Apparatus according to any preceding claim wherein the applicator includes a topical agent.

9. Apparatus according to any preceding claim, wherein a plurality of light emitters are included.

10. Apparatus according to any preceding claim, wherein the light emitter is a light emitting diode (LED) or diode laser.

11. Apparatus according to any preceding claim, wherein the wavelength of light emitted from the light emitters is substantially in the range 400 nm to 1000 nm.

12. Apparatus according to any preceding claim, wherein the wavelength of light emitted from the light emitters is substantially in the range 400 nm to 450 nm and/or 570 nm to 590 nm.

13. Apparatus according to any preceding claim, wherein the wavelength of light emitted from the light emitters is substantially in the range 570 nm to 590 nm.

14. Apparatus according to any preceding claim, wherein the light emitter is received in the applicator.

15. Apparatus according to any preceding claim, wherein the controller is provided in a housing.

16. Apparatus according to any preceding claim, further including a power source.

17. Apparatus according to claim 16, wherein the power source is one or more batteries, preferably one or more rechargeable batteries.

18. Apparatus according to any preceding claim, wherein the controller is arranged to control the period of time that light is emitted from the light emitter for one treatment period.

19. Apparatus according to claim 18, wherein the duration of light emitted from the light emitter is substantially in the range 1 minute to 1 hour.

20. Apparatus according to claim 18, wherein the duration of light emitted from the light emitter is substantially in the range 20 to 40 minutes.

21. Apparatus according to claim 18, wherein the duration of light emitted from the light emitter is about 30 minutes.

22. Apparatus according to any of claims 19 to 21, wherein the controller is configured to permit variable selection of light duration within said range and/or inhibit the duration of the period of light emitted above a predetermined time.

23. A method of cosmetically improving the appearance of skin, the method comprising providing apparatus according to any of claims 1 to 22 and directing light of a predetermined wavelength toward a target area of skin in accordance with a predetermined delivery regime.

24. A method according to claim 23 for the treatment of acne vulgaris in relation to skin

25. A method of cosmetically improving the appearance of teeth, the method comprising providing apparatus according to any of claims 1 to 22 and directing light of a predetermined wavelength toward a target area of the teeth in accordance with a predetermined delivery regime.

26. A method according to claim 23 or 25, wherein the period that light is directed towards the skin or teeth is substantially in the range 1 minute to 1 hour.

27. A method according to claim 26, wherein the period that light is directed towards the skin is substantially in the range 20 to 40 minutes.

28. A method according to claim 26, wherein the period that light is directed towards the skin or teeth is about 30 minutes.

29. A method according to any of claims 23 to 28, wherein the wavelength of light directed towards the skin surface or teeth is substantially in the range 400 nm to 1000 nm.

30. A method according to claim 29, wherein the wavelength of light directed to the skin surface is substantially in the range 400 nm to 450 nm and/or 570 nm to 590 nm.

31. A method according to claim 29, wherein the wavelength of light directed to the skin surface is substantially in the range 570 nm to 590 nm.

32. Apparatus substantially as herein described with reference to the accompanying drawings.
